(19) United States
(12) Patent Application Publication
(10) Pub. No.: US 2025/0248641 A1
(43) Pub. Date: Aug. 7, 2025

(54) INFORMATION PROCESSING APPARATUS, METHOD, AND PROGRAM

(71) Applicant: FUJIFILM CORPORATION, Tokyo (JP)

(72) Inventor: Ryota MASE, Tokyo (JP)

(21) Appl. No.: 18/977,961

(22) Filed: Dec. 12, 2024

(30) Foreign Application Priority Data
Feb. 2, 2024 (JP) 2024-015112

(51) Int. Cl. A61B 5/00; A61B 5/11; A61B 5/398; G16H 50/20

(52) U.S. Cl. CPC A61B 5/4082; A61B 5/1123; A61B 5/398; G16H 50/20

(57) ABSTRACT

A processor is configured to specify a first section and a second section in which a detection target of movement of a subject is different, based on a measurement value of the movement of the subject measured by a device that is mountable on the subject; acquire at least one feature amount representing a feature of a disease related to at least one of cognition or motion based on each of a measurement value of the first section and a measurement value of the second section; and acquire a determination result of the disease based on the feature amount and a predetermined determination criterion.

### CROSS REFERENCE TO RELATED APPLICATIONS

The present application claims priority from Japanese Patent Application No. 2024-015112, filed on Feb. 2, 2024, the entire disclosure of which is incorporated herein by reference.

### BACKGROUND OF THE INVENTION

### Technical Field

The present disclosure relates to an information processing apparatus, method, and program.

### Related Art

With an advent of a full-fledged aging society, an importance of accurately predicting a progression of a disease related to the cognition such as dementia or Parkinson's disease or a disease related to the motion and establishing an optimal treatment policy according to the prediction is increasing. For example, JP2019-531569A proposes a method of determining a disease of cognition and motion by causing a subject to perform a motion such as drawing with a hand or pressing a figure using a mobile device and comparing measurement data obtained as a result of the motion with a reference value. In addition, JP2021-029692A proposes a method of determining whether or not a subject corresponds to mild dementia by determining factor information such as an estimated value of a Mini Mental State Examination (MMSE) score causing dementia and an estimated value of a brain glucose metabolism rate, and using data acquired by a sensor worn by the subject and the factor information. In JP2021-029692A, the factor information is acquired by a regression model generated by machine learning using sensor data and attribute data (whether the subject is healthy or has mild dementia) as explanatory variables and the factor information as a response variable.

In the method described in JP2019-531569A, it is necessary for the subject to perform a motion such as drawing, so that the burden on the subject is large. In the method disclosed in JP2021-029692A, it is necessary to determine the factor information in order to obtain a determination result, and thus a processing cost is required. In addition, in the method described in JP2021-029692A, it is necessary to perform a two-stage determination process of determining the factor information for determining mild cognitive impairment. Therefore, the obtained final determination result of the mild cognitive impairment is likely to include an error, and as a result, there is a possibility that the determination accuracy is decreased.

### SUMMARY OF THE INVENTION

The present disclosure has been made in view of the above circumstances, and an object of the present disclosure is to enable determination of at least one of a disease of cognition or a disease of motion to be simply and accurately performed.

An information processing apparatus according to the present disclosure comprises at least one processor, in which the processor is configured to specify a first section and a second section in which a detection target of movement of a subject is different, based on a measurement value of the movement of the subject measured by a device that is mountable on the subject; acquire at least one feature amount representing a feature of a disease related to at least one of cognition or motion based on each of a measurement value of the first section and a measurement value of the second section; and acquire a determination result of the disease based on the feature amount and a predetermined determination criterion.

In the information processing apparatus according to the present disclosure, the first section may be a section in which movement of an entire body of the subject is a detection target, and the second section is a section in which movement of a head of the subject is a detection target.

In the information processing apparatus according to the present disclosure, the measurement value may include a change in an acceleration of the subject in a vertical downward direction, and the processor is configured to derive landing information indicating that the subject has landed during movement based on the change in the acceleration, and specify a walking section of the subject as the first section and specify a non-walking section of the subject as the second section based on continuity of the landing information.

In the information processing apparatus according to the present disclosure, the measurement value may include an acceleration in a front-rear direction of a head in the second section, and the processor is configured to acquire the feature amount representing the feature of the disease related to the cognition based on the acceleration in the front-rear direction of the head in the second section.

In the information processing apparatus according to the present disclosure, the measurement value may include a rotation speed of a head in the second section, and the processor is configured to acquire the feature amount representing the feature of the disease related to the motion based on the rotation speed of the head in the second section.

In the information processing apparatus according to the present disclosure, the measurement value may include movement in a left-right direction of the subject in the first section, and the processor is configured to acquire the feature amount representing the feature of the disease related to the cognition based on the movement in the left-right direction of the subject in the first section.

In the information processing apparatus according to the present disclosure, the measurement value may include movement of the subject in a front-rear direction in the first section, and the processor is configured to acquire the feature amount representing the feature of the disease related to the motion based on the movement of the subject in the front-rear direction in the first section.

In the information processing apparatus according to the present disclosure, the processor may be configured to derive the determination criterion.

In the information processing apparatus according to the present disclosure, the determination criterion may be a reference value for distinguishing between the disease and a non-disease.

In the information processing apparatus according to the present disclosure, the determination criterion may be a feature amount distribution estimated based on a plurality of the feature amounts representing the feature of the disease.

In the information processing apparatus according to the present disclosure, the determination criterion may be an identification model that has been trained to output a score representing a possibility of the disease in response to an input of the feature amount.

In the information processing apparatus according to the present disclosure, the disease may be at least one of mild cognitive impairment, dementia, or Parkinson's disease.

In the information processing apparatus according to the present disclosure, the device may include an acceleration sensor and an angular velocity sensor.

In the information processing apparatus according to the present disclosure, the device may include an electrooculography sensor.

An information processing method according to the present disclosure comprises, via a computer: specifying a first section and a second section in which a detection target of movement of a subject is different, based on a measurement value of the movement of the subject measured by a device that is mountable on the subject; acquiring at least one feature amount representing a feature of a disease related to at least one of cognition or motion based on each of a measurement value of the first section and a measurement value of the second section; and acquiring a determination result of the disease based on the feature amount and a predetermined determination criterion.

An information processing program according to the present disclosure causing a computer to execute: a procedure of specifying a first section and a second section in which a detection target of movement of a subject is different, based on a measurement value of the movement of the subject measured by a device that is mountable on the subject; a procedure of acquiring at least one feature amount representing a feature of a disease related to at least one of cognition or motion based on each of a measurement value of the first section and a measurement value of the second section; and a procedure of acquiring a determination result of the disease based on the feature amount and a predetermined determination criterion.

According to the present disclosure, it is possible to simply and accurately determine a brain disease.

### BRIEF DESCRIPTION OF THE DRAWINGS

FIG. 1 is a diagram showing a schematic configuration of a disease detection system to which an information processing apparatus according to the present embodiment is applied.

FIG. 2 is a diagram showing a hardware configuration of the glasses-type device according to the present embodiment.

FIG. 3 is a diagram showing a hardware configuration of an analysis server that is the information processing apparatus according to the present embodiment.

FIG. 4 is a functional configuration diagram of the analysis server that is the information processing apparatus according to the present embodiment.

FIG. 5 is a diagram showing a detection result of a landing candidate.

FIG. 6 is a diagram for describing derivation of a distribution of a feature amount to which a kernel density estimation method is applied.

FIG. 7 is a diagram showing an identification model.

FIG. 8 is a diagram for describing determination of a disease in a case where a determination criterion is a representative value of a feature amount.

FIG. 9 is a diagram for describing determination of a disease in a case where a determination criterion is a distribution of a feature amount.

FIG. 10 is a diagram showing a display screen of a determination result.

FIG. 11 is a flowchart showing a process performed in the present embodiment.

FIG. 12 is a diagram for describing correction of a detection result of a landing candidate.

### DETAILED DESCRIPTION

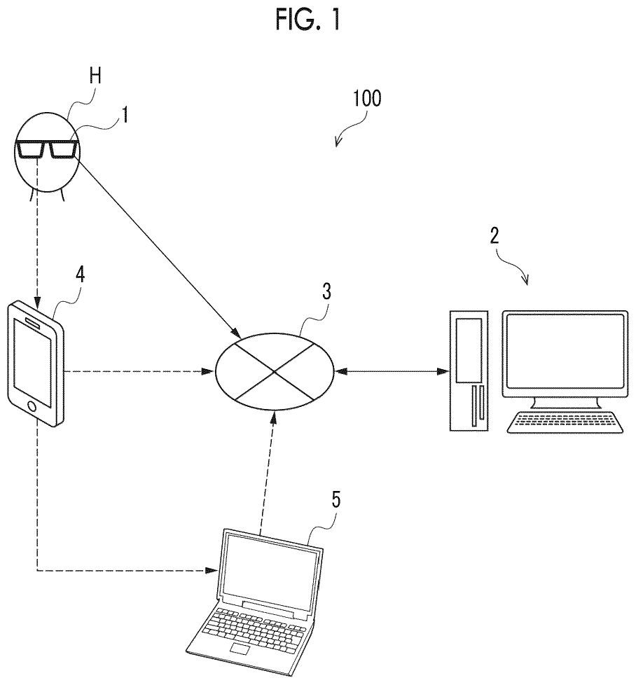

In the following, embodiments of the present disclosure will be explained with reference to the drawings. First, a configuration of a disease detection system to which an information processing apparatus according to the present embodiment is applied will be described. FIG. 1 is a diagram showing a schematic configuration of a disease detection system. In a disease detection system 100 shown in FIG. 1, a glasses-type device 1 and an analysis server 2 which is an information processing apparatus according to the present embodiment are connected in a communicable state via a network 3. The disease detection system 100 may further comprise a mobile terminal 4 such as a smartphone or a tablet terminal and a general-purpose computer 5 in addition to the analysis server 2 and the glasses-type device 1.

In the present embodiment, the analysis server 2 derives a determination result of a disease related to at least one of cognition or motion of a subject H. In the present embodiment, the analysis server 2 acquires the determination result of the disease related to both the cognition and the motion of the subject H. Specifically, a determination result of mild cognitive impairment (MCI), which is a cognitive disease, and a determination result of Parkinson's disease, which is a motion disease, are acquired.

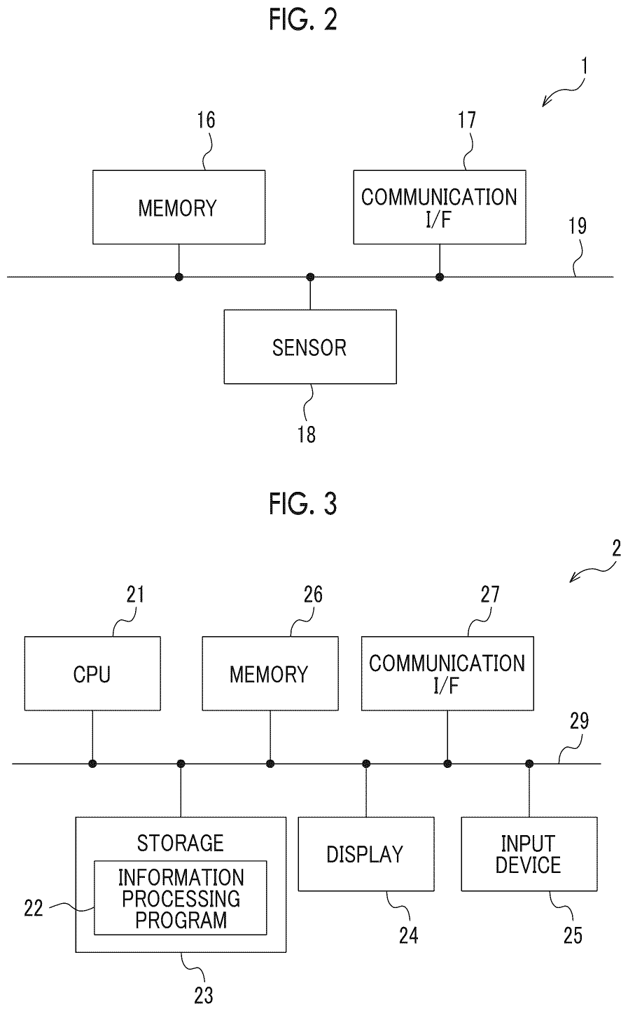

First, the glasses-type device 1 will be described. The glasses-type device 1 is a wearable device that is worn by the subject H that is a detection target of a disease and can measure movement of the subject H. FIG. 2 is a diagram showing a hardware configuration of the glasses-type device 1. As shown in FIG. 2, the glasses-type device 1 comprises a memory 16 as a temporary storage region, a communication interface (I/F) 17, and a sensor 18. The memory 16, the communication I/F 17, and the sensor 18 are connected to a bus 19. Examples of such a glasses-type device include “JINS MEME” (registered trademark) manufactured by JINS HOLDINGS Inc. The glasses-type device 1 may have a CPU, a screen that performs various types of display, and the like, as in the case of smart glasses.

The sensor 18 is provided at a space between the eyebrows and a nose rest portion of the glasses-type device 1 and detects the movement of the subject H. Specifically, the sensor 18 is a 6-axis sensor having a 3-axis acceleration sensor and a 3-axis angular velocity sensor so as to be able to detect 3-axis acceleration and angular velocity around 3-axis. The sensor 18 may be a 9-axis sensor further having a 3-axis earth axis sensor in addition to the 3-axis acceleration sensor and the 3-axis angular velocity sensor. The sensor 18 may include an electrooculography sensor that detects an electrooculography potential.

In the present embodiment, the subject H is caused to wear the glasses-type device 1, and the subject H is allowed to act freely. Then, the sensor 18 measures the movement of the subject H during the free action of the subject H and acquires the measurement value. In the present embodiment, since the sensor 18 is a 6-axis sensor, six measurement values are acquired during the free action of the subject H. Therefore, in the present embodiment, the sensor 18 acquires the time-series measurement values over a period in which the subject H freely acts.

The three axes measured by the sensor 18 are a front-rear direction, a left-right direction, and an up-down direction of the subject H, and measurement values of accelerations in the front-rear direction, the left-right direction, and the up-down direction of the subject H are acquired by the acceleration sensor. The measurement values of the accelerations of the subject H in the front-rear direction, the left-right direction, and the up-down direction represent the movements of the subject H in the front-rear direction, the left-right direction, and the up-down direction. In addition, the angular velocity sensor acquires a measurement value of the angular velocity by rotation around an axis extending in the front and rear directions, rotation around an axis extending in the left and right directions, and rotation around an axis extending in the up and down directions of the subject H. The measurement value of the angular velocity represents rotation speeds around an axis extending in the front and rear directions, around an axis extending in the left and right directions, and around an axis extending in the up and down directions of the subject H.

In the present embodiment, data of the measurement value acquired by the sensor 18 is temporarily stored in the memory 16, and is transmitted to the analysis server 2 from the communication I/F 17 via the network 3 as a measurement value after the measurement period ends.

The communication I/F 17 may be connected to the network 3 in a wireless manner to perform wireless communication, or may perform short-range wireless communication such as Bluetooth (registered trademark). A plurality of communication I/Fs 17 may be provided so that both wireless communication and short-range wireless communication can be performed. In a case where the communication I/F 17 performs the short-range wireless communication, the measurement value acquired by the glasses-type device 1 is transmitted to, for example, the mobile terminal 4 of the subject H or the like and is transmitted from the mobile terminal 4 to the analysis server 2 via the network 3. Alternatively, the measurement value is transmitted from the mobile terminal 4 to the computer 5 by short-range wireless communication or wired connection, and is transmitted from the computer 5 to the analysis server 2 via the network 3. In a case where the measurement value is transmitted to the mobile terminal 4 or the computer 5, the measurement value may be confirmed on the mobile terminal 4 or the computer 5.

The measurement value acquired by the sensor 18 includes a fine vibration component that can be regarded as noise, in addition to a value indicating movement to be acquired. Therefore, in the glasses-type device 1, the sensor 18 may perform noise removal processing on the acquired data and may transmit the data from which noise is removed to the analysis server 2 as a measurement value. Examples of the noise removal processing include filtering processing using a low-pass filter, but the noise removal processing is not limited thereto.

The network 3 may be a local area network (LAN) that locally connects the glasses-type device 1 and the analysis server 2, or may be a wide area network (WAN) that connects the analysis server 2 and the glasses-type device 1 over a wide area through a public line network or a dedicated line network.

Next, the analysis server 2 will be described. FIG. 3 is a diagram showing a hardware configuration of an analysis server 2 that is an information processing apparatus. As shown in FIG. 3, the analysis server 2 includes a CPU 21, a non-volatile storage 23, and a memory 26 serving as a temporary storage region. In addition, the analysis server 2 includes a display 24 such as a liquid crystal display, an input device 25 including a pointing device such as a keyboard and a mouse, and a communication I/F 27 connected to the network 3. The CPU 21, the storage 23, the display 24, the input device 25, the memory 26, and the communication I/F 27 are connected to a bus 29. The CPU 21 is an example of a processor according to the present disclosure.

The storage 23 is realized by a hard disk drive (HDD), an SSD, a flash memory, and the like. An information processing program 22 is stored in the storage 23 as the storage medium. The CPU 21 reads out an information processing program 22 from the storage 23, loads the information processing program 22 into the memory 26, and executes the loaded information processing program 22.

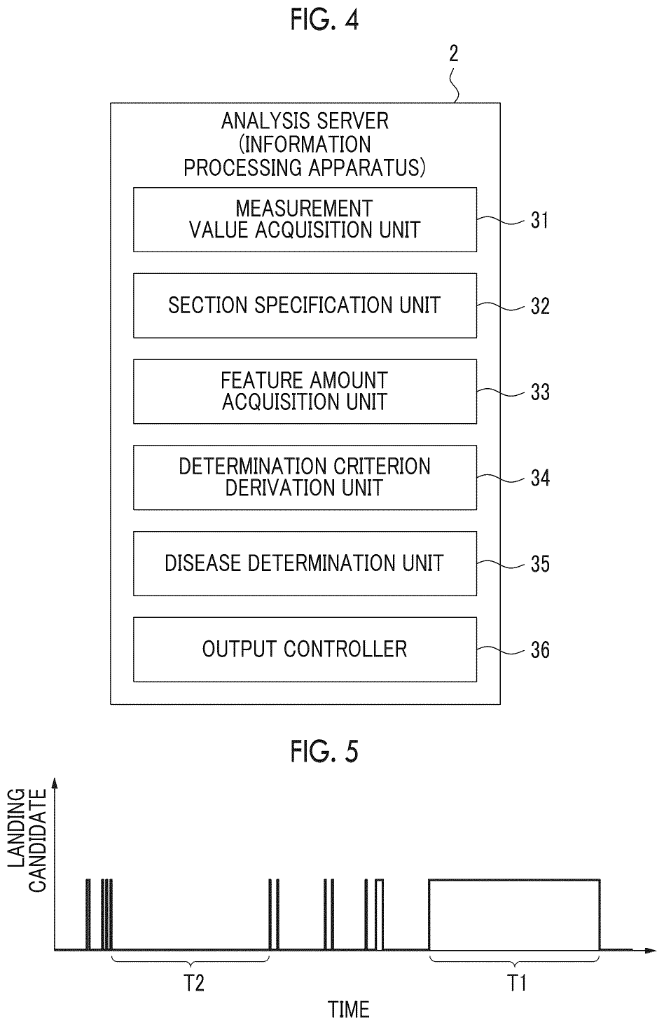

Next, a functional configuration of the analysis server 2, which is the information processing apparatus according to the present embodiment, will be described. FIG. 4 is a diagram showing a functional configuration of the analysis server 2 that is the information processing apparatus according to the present embodiment. As shown in FIG. 4, the analysis server 2 comprises a measurement value acquisition unit 31, a section specification unit 32, a feature amount acquisition unit 33, a determination criterion derivation unit 34, a disease determination unit 35, and an output controller 36. Then, the CPU 21 executes the information processing program 22, so that the CPU 21 functions as the measurement value acquisition unit 31, the section specification unit 32, the feature amount acquisition unit 33, the determination criterion derivation unit 34, the disease determination unit 35, and the output controller 36.

The measurement value acquisition unit 31 acquires the measurement value transmitted from the glasses-type device 1 via the network 3 through the communication I/F 27.

The section specification unit 32 specifies the first section and the second section in which the detection target of the movement of the subject His different from each other, based on the measurement value acquired by the measurement value acquisition unit 31. In the present embodiment, the first section is a section in which the movement of the entire body of the subject H is a detection target, and the second section is a section in which the movement of the head of the subject His a detection target. The movement of the entire body of the subject H occurs when the subject H is moving, particularly during walking. The movement of the head of the subject H occurs when the subject H is not moving, particularly when the subject H is not walking. Therefore, in the present embodiment, the first section is set as a walking section, and the second section is set as a non-walking section.

In the present embodiment, the measurement value measured by the glasses-type device 1 includes the acceleration of the subject H in the up-down direction as described above. Here, in a case where a person is walking, a large change in acceleration occurs in a vertical downward direction in a case where one foot lands on the ground. Therefore, in the present embodiment, the section specification unit 32 detects, as the landing candidate, a time point at which a change in the acceleration in the vertical downward direction is equal to or greater than a predetermined threshold value based on the acceleration in the up-down direction included in the measurement value.

FIG. 5 is a diagram showing a detection result of the landing candidate. In FIG. 5, a lateral axis indicates a time at which the measurement value is acquired, and a vertical axis indicates an indicator indicating that the landing candidate is detected. As shown in FIG. 5, the detection result of the landing candidate includes a section T1 in which the landing candidate is continuously detected over a period in which the landing candidate is present, and a section T2 in which the landing candidate is not detected at all over the period in which the landing candidate is present.

Here, in a case where the person is walking, landing is continuously performed, so that the landing candidate is continuously detected. As shown in FIG. 5, the section specification unit 32 specifies a section T1 in which the landing candidate is continuously detected over a predetermined period as a walking section in all periods in which the measurement value is acquired. In addition, as shown in FIG. 5, the section specification unit 32 specifies the section T2 in which the landing candidate is not detected at all over a predetermined period as a non-walking section. In the present embodiment, the measurement value acquisition unit 31 acquires the measurement value in time series. Therefore, the section specification unit 32 specifies the start time point and the end time point of the section T1 as the walking section, and specifies the start time point and the end time point of the section T2 as the non-walking section. The walking section and the non-walking section may be specified using the start time point and a length of a time at which the section ends, instead of specifying the start time point and the end time point.

The feature amount acquisition unit 33 acquires at least one feature amount representing a feature of a disease related to at least one of cognition or motion based on each of the measurement value of the first section and the measurement value of the second section. Specifically, a plurality of feature amounts representing the features of the MCI and the Parkinson's disease are acquired based on each of the walking section and the non-walking section specified by the section specification unit 32 as described above.

Here, as the movement of the head, the patient with MCI tends to have a larger variation in the shake in the front-rear direction than the healthy person. A patient with Parkinson's disease tends to have a slower rotational movement of the head, such as shaking the head sideways, than a healthy person.

In addition, as the movement of the entire body, the patient with MCI tends to have a worse balance in the left-right direction than the healthy person. Since the patient with Parkinson's disease has a slow movement of the entire body, the movement in the front-rear direction tends to be smaller than that of a healthy person.

Therefore, in the non-walking section in which the movement of the head of the subject His a detection target, the feature amount acquisition unit 33 acquires, as the feature amount, information related to the acceleration in the front-rear direction of the subject H and information related to the rotation speed. As the information related to the acceleration in the front-rear direction, for example, statistical information of an acceleration component in the front-rear direction in the non-walking section included in the measurement value can be used. As the statistical information of the acceleration component in the front-rear direction, a statistical value related to the variation of values such as the standard deviation and the interquartile range of the acceleration component in the front-rear direction can be used. In addition, as the statistical information of the acceleration component in the front-rear direction, a statistical value representing the acceleration component, such as a maximum value, a minimum value, an average value, a median value, a most frequent value, and a quartile of the acceleration component in the front-rear direction, can be used. In addition, as the statistical information of the acceleration component in the front-rear direction, information related to a shape of a distribution of the acceleration component, such as a sharpness and a skewness of the acceleration component in the front-rear direction, can be used. The statistical information of the acceleration component in the front-rear direction is not limited thereto.

As the information related to the rotation speed, for example, statistical information of angular velocity components of the subject H in the front-rear direction and the left-right direction in the non-walking section can be used. As the statistical information of the angular velocity components of the subject H in the front-rear direction and the left-right direction, statistical values such as the average value, the median value, and the quartile of the angular velocity components in the rotation around an axis extending in the front and rear directions, rotation around the axis extending in the left and right directions, and rotation around the axis extending in the up and down directions of the subject H, and information such as the 90 percentile, the 95 percentile, and the 97 percentile can be used, but the present disclosure is not limited thereto. The percentile is a percentage representing a position of a certain measurement value in the entire measurement values.

On the other hand, in the walking section in which the movement of the entire body of the subject His a detection target, the feature amount acquisition unit 33 acquires, as the feature amount, information related to the movement in the left-right direction of the subject H and information related to the movement in the front-rear direction of the subject H. As the information related to the movement in the left-right direction, for example, statistical information of the acceleration component in the left-right direction in the walking section can be used. As the statistical information of the acceleration components in the left-right direction, an indicator such as a difference between average values of the acceleration components in each of the left and right directions, a difference between cumulative values of the acceleration components in each of the left and right directions, or a difference between cumulative values of the acceleration components in each of the left and right directions at a timing at which a large change occurs in the acceleration component in the vertical direction (that is, a timing at which the landing candidate appears) can be used. In addition, as the statistical information of the acceleration component in the left-right direction, a statistical value related to the variation of values such as the standard deviation and the interquartile range of the acceleration component in the left-right direction in the walking section can be used. In addition, as the statistical information of the acceleration component in the left-right direction, a statistical value representing the acceleration component, such as a maximum value, a minimum value, an average value, a median value, a most frequent value, and a quartile of the acceleration component in the left-right direction, can be used. In addition, as the statistical information of the acceleration component in the left-right direction, information related to a shape of a distribution of the acceleration component value, such as a sharpness and a skewness of the acceleration component in the left-right direction, can be used. As the information related to the movement in the front-rear direction, the same statistical information as described above for the acceleration component in the front-rear direction can be used. The statistical information is not limited to the above.

In a case where the sensor 18 includes an electrooculography sensor, the measurement value acquired by the measurement value acquisition unit 31 includes a measurement value of the electrooculography sensor. Here, a patient with Parkinson's disease tends to have a smaller number of blinks than a healthy person. Therefore, in a case where the sensor 18 includes the electrooculography sensor, the feature amount acquisition unit 33 acquires a time point at which a blink occurs from a change pattern of an electrooculography potential, and acquires the number of blinks per unit time as the feature amount.

The determination criterion derivation unit 34 derives a determination criterion used in a case where a disease determination unit 35, which will be described later, acquires a determination result of a disease, in advance, and stores the derived determination criterion in the storage 23. In a case of deriving the determination criterion, the determination criterion derivation unit 34 acquires the same feature amount as the feature amount acquisition unit 33 from the measurement values of a large number of subjects H including a healthy person, a patient with MCI, and a patient with Parkinson's disease. That is, in the non-walking section in which the movement of the head of the subject His a detection target, the determination criterion derivation unit 34 acquires, as the feature amount, information related to the acceleration in the front-rear direction of a large number of subjects H and information related to the rotation speed. In addition, in the walking section in which the movement of the entire body of a subject His the detection target, the determination criterion derivation unit 34 acquires, as the feature amount, information related to the movement in the left-right direction of the large number of subject H and information related to the movement in the front-rear direction of the large number of subject H. The determination criterion derivation unit 34 acquires the feature amount in association with the attribute of the patient, that is, the attribute of each of the healthy person, the MCI, and the Parkinson's disease.

The determination criterion derivation unit 34 derives a determination criterion for each acquired feature amount in association with the attribute. For example, the determination criterion derivation unit 34 derives a representative value of the feature amount for each attribute as the determination criterion. For example, in the non-walking section in which the movement of the head is a detection target, the determination criterion derivation unit 34 derives, as the determination criterion of the MCI, a representative value of a feature amount which is information related to the acceleration in the front-rear direction of a large number of patients with MCI. In addition, in which the movement of the entire body is a detection target, the determination criterion derivation unit 34 derives, as the determination criterion for MCI in the walking section, a representative value of a feature amount which is information related to the movement in the left-right direction of a large number of patients with MCI.

In addition, in the non-walking section in which the movement of the head is a detection target, the determination criterion derivation unit 34 derives, as the determination criterion for Parkinson's disease, a representative value of a feature amount which is information related to rotation speeds of a large number of patients with Parkinson's disease. In addition, in the walking section in which the movement of the entire body is a detection target, the determination criterion derivation unit 34 derives, as the determination criterion for Parkinson's disease, a representative value of a feature amount which is information related to the movement in the front-rear direction of a large number of patients with Parkinson's disease.

Regarding the healthy person, for the non-walking section, the determination criterion derivation unit 34 derives, as the determination criterion for the healthy person, a representative value of a feature amount which is information related to the acceleration in the front-rear direction of a large number of healthy persons, and a representative value of a feature amount which is information related to the rotation speed of a large number of healthy persons. For the walking section, the determination criterion derivation unit 34 derives, as the determination criterion of the healthy person, each of a representative value of a feature amount which is information related to the movement in the left-right direction of a large number of healthy persons and a representative value of a feature amount which is information related to the movement in the front-rear direction of a large number of healthy persons.

As the representative value, a maximum value, a minimum value, an average value, a median value, a most frequent value, a quartile, and the like can be used, but the present disclosure is not limited thereto.

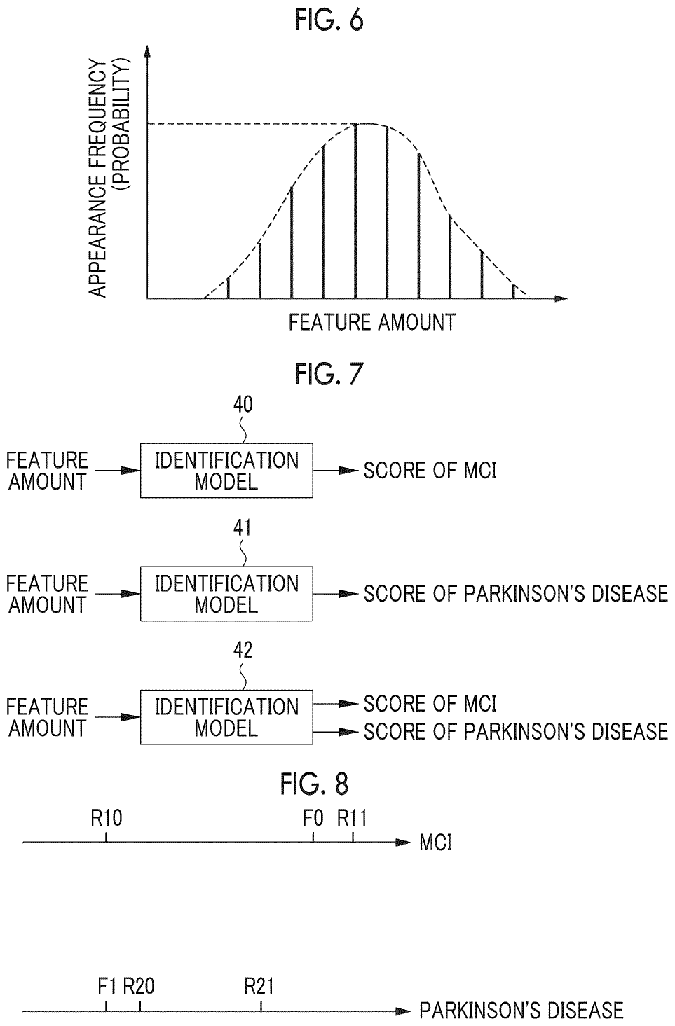

In addition, as the determination criterion, a distribution of the feature amount can be used instead of the representative value of the feature amount. The distribution of the feature amount can be obtained by applying, for example, a kernel density estimation method to a large number of feature amounts. The kernel density estimation is a method of estimating a distribution of the feature amount such that a change in the distribution is smooth by applying a kernel function to a distribution of raw data of the feature amount, such as a histogram of the feature amount. FIG. 6 is a diagram for describing derivation of a distribution of the feature amount to which a kernel density estimation method is applied. In FIG. 6, the lateral axis represents a value of the feature amount, and the vertical axis represents an appearance frequency of the feature amount. By applying the kernel density estimation to the distribution of the raw data of the feature amount indicated by the solid line in FIG. 6, the distribution of the feature amount indicated by the broken line in FIG. 6 can be derived. In the distribution of the feature amount, the vertical axis represents the appearance probability. The probability may be derived by normalizing the distribution of the determination criterion by setting a peak of the determination criterion to, for example, 0.8.

In a case where the distribution of the feature amount is used as the determination criterion, in the non-walking section, the determination criterion derivation unit 34 derives, as the determination criterion for the MCI, a distribution of the feature amount which is information related to the acceleration in the front-rear direction of a large number of patients with MCI. In addition, in the walking section, the determination criterion derivation unit 34 derives, as the determination criterion for the MCI, a distribution of the feature amount which is information related to the movement in the left-right direction of a large number of patients with MCI. In addition, in the non-walking section, the determination criterion derivation unit 34 derives, as the determination criterion for the Parkinson's disease, a distribution of the feature amount which is information related to rotation speed of a large number of patients with Parkinson's disease. In addition, in the walking section, the determination criterion derivation unit 34 derives, as the determination criterion for the Parkinson's disease, a distribution of the feature amount which is information related to the movement in the front-rear direction of a large number of patients with Parkinson's disease.

For the healthy person, for the non-walking section, the determination criterion derivation unit 34 derives, as the determination criterion of the healthy person, a distribution of the feature amount which is information related to the acceleration in the front-rear direction of a large number of healthy persons and a distribution of the feature amount which is information related to the rotation speed of a large number of healthy persons. For the walking section, the determination criterion derivation unit 34 derives, as the determination criterion of the healthy person, a distribution of the feature amount which is information related to the movement in the left-right direction of a large number of the healthy persons and a distribution of the feature amount which is information related to the movement in the front-rear direction of a large number of the healthy persons, respectively.

In addition, in the non-walking section, as the determination criterion for the Parkinson's disease, a distribution of the feature amount which is the information related to the acceleration in the front-rear direction of a large number of patients with Parkinson's disease may be derived in addition to the distribution of the feature amount which is the information related to the rotation speed of a large number of patients with Parkinson's disease. In addition, in the walking section, as the determination criterion for the Parkinson's disease, a distribution of the feature amount which is the information related to the movement in the left-right direction of a large number of patients with Parkinson's disease may be derived in addition to the distribution of the feature amount which is the information related to the movement in the front-rear direction of a large number of patients with Parkinson's disease. In addition, in the non-walking section, as the determination criterion for the MCI, a distribution of the feature amount which is information related to a rotation speed of a large number of patients with MCI may be derived in addition to the distribution of the feature amount which is information related to the acceleration in the front-rear direction of a large number of patients with MCI. In addition, in the walking section, as the determination criterion for the MCI, a distribution of the feature amount which is the information related to the movement in the front-rear direction of the large number of patients with MCI may be derived in addition to the distribution of the feature amount which is the information related to the movement in the left-right direction of the large number of patients with MCI.

In addition, as the determination criterion, an identification model for identifying a patient with MCI and a healthy person and an identification model for identifying a patient with Parkinson's disease and a healthy person can be used. For example, for the identification model for identifying the patient with MCI and the healthy person, the identification model can be constructed by subjecting the neural network to machine learning such that the feature amount of the subject His input and a score indicating that the subject H is the MCI is output, using the feature amount acquired from the patient with MCI and the feature amount acquired from the healthy person as training data, respectively. The score takes, for example, a value of 0 to 1, and the closer the score is to 1, the higher the possibility of MCI. The feature amount used in a case of constructing the identification model for determining the MCI is information related to the acceleration in the front-rear direction in the non-walking section and information related to the movement in the left-right direction in the walking section.

The identification model for identifying the patient with Parkinson's disease and the healthy person can be constructed by subjecting a neural network to machine learning such that the feature amount of the subject H is input and a score indicating that the subject H is the Parkinson's disease is output, using the feature amount acquired from the patient with Parkinson's disease and the feature amount acquired from the healthy person as training data, respectively. The score takes, for example, a value of 0 to 1, and the closer the score is to 1, the higher the possibility of Parkinson's disease. The feature amount used in a case of constructing the identification model for determining the Parkinson's disease is information related to the rotation speed in the non-walking section and information related to the movement in the front-rear direction in the walking section.

In a case where the score is derived for the healthy person, the score indicating that the subject H is the patient with MCI or the score indicating that the subject H is the patient with Parkinson's disease may be subtracted from 1.

As a machine learning method, any method such as linear discriminant analysis, support vector machine, and random forest can be used. As shown in FIG. 7, as the determination criteria, the identification model 40 for identifying the patient with MCI and the identification model 41 for identifying the patient with Parkinson's disease may be separately derived, but only one identification model 42 that outputs both the score indicating that the subject H is the patient with MCI and the score indicating that the subject H is the patient with Parkinson's disease may be constructed.

In addition, in a case where the feature amount is acquired in a case of deriving the determination criterion, the subject from which the feature amount is acquired may suffer from both dementia and Parkinson's disease. For such a subject H, the feature amount may have a tendency different from that of the subject suffering from only dementia and the subject suffering from only Parkinson's disease. Therefore, in a case where the identification model is derived as a determination criterion, only the feature amount acquired from the subject suffering from only dementia and the subject suffering from only Parkinson's disease may be used.

The disease determination unit 35 acquires a determination result of whether the subject His a healthy person, MCI, or Parkinson's disease based on the feature amount acquired by the feature amount acquisition unit 33 and the determination criterion derived by the determination criterion derivation unit 34. In a case where the determination criterion is the representative value of the feature amount, the disease determination unit 35 compares the representative value for the three attributes of the healthy person, the MCI, and the Parkinson's disease with the feature amount acquired by the feature amount acquisition unit 33 for the subject H. Specifically, among the determination criteria for each attribute, such as the healthy person, the MCI, and the Parkinson's disease, an attribute that is the determination criterion closest to the feature amount of the subject His acquired as the determination result of the disease of the subject H.

FIG. 8 is a diagram for describing determination of a disease in a case where the determination criterion is the representative value of the feature amount. As shown in FIG. 8, for the MCI, a feature amount F0 of the subject H, a representative value R10 which is the determination criterion for being the healthy person, and a representative value R11 which is the determination criterion for being the patient with MCI are compared with each other. In this case, the feature amount F0 of the subject His close to the representative value R11. In addition, for the Parkinson's disease, a feature amount F1 of the subject H, a representative value R20 which is the determination criterion for being the healthy person, and a representative value R21 which is the determination criterion for being the patient with Parkinson's disease are compared with each other. In this case, the feature amount F1 of the subject His close to the representative value R20. Therefore, the disease determination unit 35 acquires a determination result that the subject H is highly likely to have MCI.

In a case where the determination criterion is the distribution of the feature amounts for each of the attributes of the healthy person, the MCI, and the Parkinson's disease, the disease determination unit 35 derives the appearance probability of the feature amount of the subject H from the distribution of the feature amounts. Then, the attribute having the highest appearance probability of the feature amount is acquired as the determination result of the disease of the subject H.

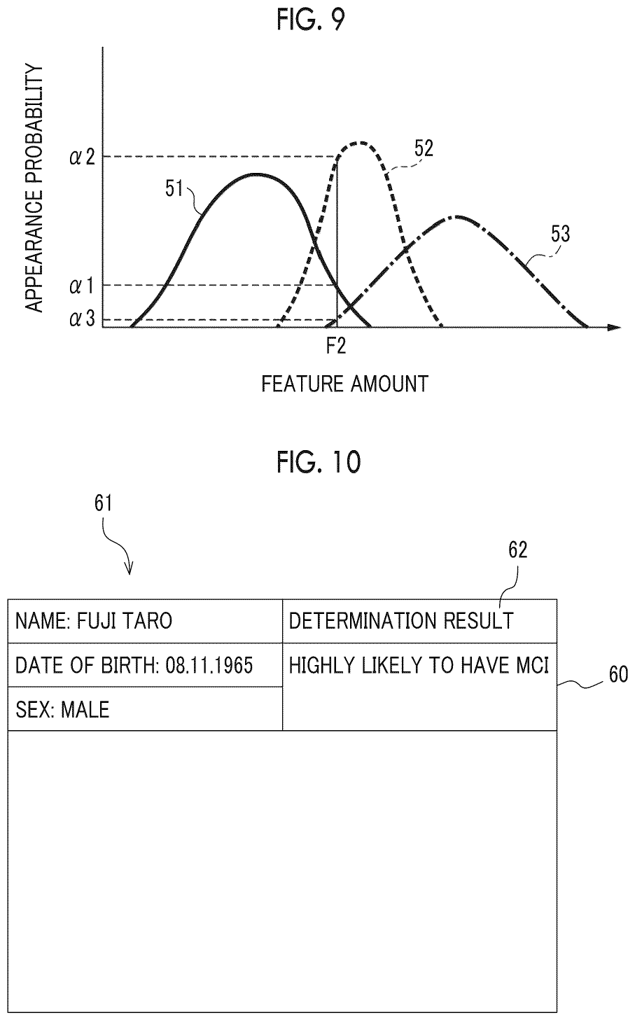

FIG. 9 is a diagram for describing determination of a disease in a case where a determination criterion is a distribution of a feature amount. As shown in FIG. 9, it is assumed that a distribution 51 indicating that the subject His a healthy person, a distribution 52 indicating that the subject H has MCI, and a distribution 53 indicating that the subject H has Parkinson's disease are acquired as determination criteria for a certain feature amount. The disease determination unit 35 applies the feature amount F2 of the subject H to the distribution of the three feature amounts to derive an appearance probability α1 indicating that the subject H is a healthy person, an appearance probability α2 indicating that the subject H has MCI, and an appearance probability α3 indicating that the subject H has Parkinson's disease. In this case, α2>α1>α3. Therefore, the disease determination unit 35 acquires a determination result that the possibility of MCI with the highest appearance probability is high for the subject H.

It should be noted that the appearance probability α1 indicating that the subject His a healthy person, the appearance probability α2 indicating that the subject H has MCI, and the appearance probability α3 indicating that the subject H has Parkinson's disease may be used as the determination result as it is.

In a case where the determination criterion is the identification model, the disease determination unit 35 inputs the feature amount of the subject H to the identification model, and acquires a score α11 indicating that the subject H has MCI and the score α12 indicating that the subject H has Parkinson's disease as the determination results, respectively. The disease determination unit 35 may acquire the attribute having a large score for the subject H as the determination result. For example, in a case where the score α12 indicating that the subject H has Parkinson's disease is larger than the score α11 indicating that the subject H has MCI, the disease determination unit 35 may acquire a determination result that the subject H is highly likely to have Parkinson's disease.

The output controller 36 displays the determination result output by the disease determination unit 35 on the display 24. FIG. 10 is a diagram showing a display screen of the determination result. As shown in FIG. 10, a display screen 60 displays subject information 61 such as a name, a gender, and a date of birth of the subject H and a determination result 62. In FIG. 10, a determination result that the subject is highly likely to have MCI is displayed. The output controller 36 may notify and display the determination result by a pop-up or the like instead of displaying the display screen 60 shown in FIG. 10. In addition, the output controller 36 may output the determination result by voice instead of or in addition to the display of the determination result. In addition, the output controller 36 may output the determination result by transmitting the determination result to an external terminal device such as a mobile terminal 4 of the subject H, instead of displaying the determination result on the display 24. In this case, the output controller 36 may output the determination result by transmitting the determination result to the terminal device by e-mail or by transmitting the determination result to an application of the terminal device. The terminal device may display the received determination result by e-mail or an application.

In the present embodiment, in a case where the representative value of the feature amount and the distribution of the feature amount are used as the determination criterion, information related to the acceleration in the front-rear direction in the non-walking section and information related to the movement in the left-right direction in the walking section are used to determine MCI. In addition, in order to determine Parkinson's disease, information related to a rotation speed in the non-walking section and information related to a movement in the front-rear direction in the walking section are used. Therefore, as the determination results of the MCI and the Parkinson's disease, the determination results based on the two determination criteria are obtained, respectively. In general, according to the disease suffered by the subject H, the determination results using the two determination criteria are the same, but the determination results using the two determination criteria may be different. In such a case, the disease determination unit 35 may acquire a determination result indicating that the determination is not possible. In this case, the output controller 36 may display the determination result indicating that the determination is not possible on the display 24. Alternatively, among the determination results based on the two determination criteria, a determination result closer to the representative value of the feature amount or a determination result with a higher appearance probability in the distribution of the feature amount may be used as the determination result of the disease of the subject H.

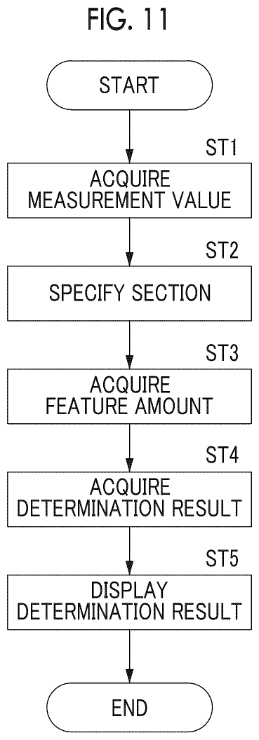

Next, processing performed in the present embodiment will be described. FIG. 11 is a flowchart showing processing performed in the present embodiment. It is assumed that the determination criterion is derived by the determination criterion derivation unit 34 and is stored in the storage 23. First, the measurement value acquisition unit 31 acquires the measurement value transmitted from the glasses-type device 1 (step ST1). Next, the section specification unit 32 specifies a first section and a second section in which the detection target of the movement of the subject His different (section specification; step ST2).

Subsequently, the feature amount acquisition unit 33 acquires at least one feature amount representing a feature of a disease related to at least one of cognition or motion based on each of the measurement value of the first section and the measurement value of the second section (step ST3). That is, a plurality of feature amounts representing the features of the MCI and the Parkinson's disease are acquired in each of the walking section and the non-walking section specified by the section specification unit 32.

Next, the disease determination unit 35 acquires the determination result of whether the subject His a healthy person, MCI, or Parkinson's disease based on the feature amount acquired by the feature amount acquisition unit 33 and the determination criterion derived by the determination criterion derivation unit 34 (step ST4). Then, the output controller 36 displays the determination result on the display 24 (step ST5), and the processing is ended.

As described above, in the present embodiment, the measurement value is acquired from the wearable device such as the glasses-type device 1 worn by the subject H, the first section and the second section in which the detection target of the movement of the subject His different are specified, at least one feature amount representing the feature of the disease related to at least one of the cognition or the motion is acquired based on each of the measurement value of the first section and the measurement value of the second section, and the determination result of the disease is acquired based on the feature amount and the predetermined determination criterion. Therefore, it is possible to acquire the measurement value necessary for the disease determination by the free action even in a case where the subject H does not perform any operation by himself/herself. Therefore, it is possible to reduce the burden on the subject H for determining the disease.

In addition, in order to obtain the determination result, it is sufficient only to specify the first section and the second section and to derive the feature amount in each section, and it is not necessary to determine the factor information as in the method described in JP2021-029692A. Therefore, the processing cost for determination is reduced, and the determination accuracy can be improved.

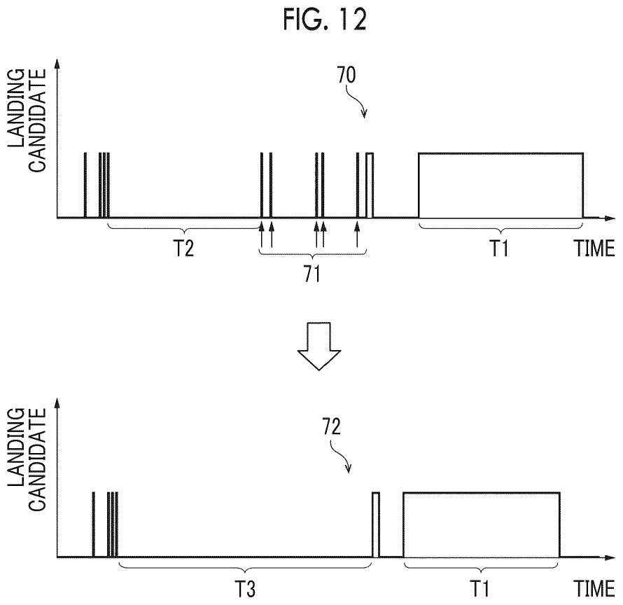

In the above-described embodiment, as shown in FIG. 5, the landing candidate detected by the section specification unit 32 includes a section in which the landing candidate randomly appears, in addition to the sections T1 and T2. Therefore, the relationship between the time point and the landing candidate shown in FIG. 5 may be displayed on the display 24, and the operator of the analysis server 2 may be allowed to receive correction of the section. FIG. 12 is a diagram for describing the correction of the section. A relationship between the time point and the landing candidate on an upper side of FIG. 12 is the same as the relationship between the time point and the landing candidate shown in FIG. 5. In the relationship 70 on the upper side of FIG. 12, there is a section in which landing candidates appear sparsely between the section T1 and the section T2. Arrows are assigned and shown to five landing candidates 71 that appear sparsely. The landing candidates that appear sparsely in this way can be regarded as some noise generated during the non-walking of the subject H.

Therefore, the operator issues an instruction to delete the five landing candidates 71 that appear sparsely in the displayed relationship 70. Accordingly, the section specification unit 32 deletes the five landing candidates 71 for which the deletion instruction is given. Then, the section T2 is widened as shown in a relationship 72 on a lower side of FIG. 12 by deleting the landing candidate 71. Therefore, the section specification unit 32 specifies the section widened by the correction as a new non-walking section T3. As a result, it is possible to more accurately specify the walking section and the non-walking section.

It should be noted that, instead of deleting the landing candidates that appear sparsely as described above, the walking section may be specified by adding the landing candidate. In addition, the walking section may be specified by deleting a section that is unnecessary as the walking section.

In addition, in the above-described embodiment, the noise removal processing may be performed on the data detected by the sensor 18 in the glasses-type device 1, but the present disclosure is not limited thereto. The measurement value may be transmitted to the analysis server 2 without performing the noise removal processing in the glasses-type device 1, and the noise removal processing of the measurement value may be performed in the analysis server 2.

In addition, in the above-described embodiment, the analysis server 2 comprises the determination criterion derivation unit 34, but the present disclosure is not limited thereto. The determination criterion derivation unit 34 may be omitted in the analysis server 2, and the analysis server 2 may acquire the determination criterion derived in the external server and store the acquired determination criterion in the storage 23.

In addition, in the above-described embodiment, the determination of MCI and the Parkinson's disease is performed, but the present disclosure is not limited thereto. In addition to MCI and Parkinson's disease, determination of dementia may be performed. Although dementia is a disease resulting from the progression of MCI, in a case where the above-described feature amount is calculated, a feature amount of a value or a distribution different from MCI is obtained. Therefore, the determination criterion derivation unit 34 may derive the determination criterion using a plurality of feature amounts for the patient with dementia and determine whether the subject H has any of MCI, dementia, or Parkinson's disease using the feature amount acquired from the measurement value acquired from the subject H and the determination criterion for dementia.

In addition, in the above-described embodiment, the measurement value of the subject H is acquired by causing the subject H to wear the glasses-type device 1, but the present disclosure is not limited thereto. A sensor may be worn on a waist or an arm of the subject H in addition to the glasses-type device 1, and a measurement value representing movement of the waist or the arm of the subject may be acquired.

In addition, in the above-described embodiment, the measurement value of the subject H is acquired by causing the subject H to wear the glasses-type device 1, but the present disclosure is not limited thereto. Instead of the glasses-type device 1, the subject H may wear an earring-type device to acquire the measurement value representing the movement of the head of the subject.

In addition, in the embodiment described above, various processors shown below can be used as the hardware structures of processing units that execute various pieces of processing, such as the measurement value acquisition unit 31, the section specification unit 32, the feature amount acquisition unit 33, the determination criterion derivation unit 34, the disease determination unit 35, and the output controller 36. As described above, the various processors include, in addition to the CPU that is a general-purpose processor which executes software (program) and functions as various processing units, a programmable logic device (PLD) that is a processor whose circuit configuration can be changed after manufacture, such as a field programmable gate array (FPGA), and a dedicated electrical circuit that is a processor having a circuit configuration which is designed for exclusive use in order to execute specific processing, such as an application specific integrated circuit (ASIC).

One processing unit may be configured by one of these various processors, or may be configured by a combination of two or more processors of the same type or different types (for example, a combination of a plurality of FPGAs or a combination of the CPU and the FPGA). A plurality of processing units may be configured with one processor.

As an example of configuring the plurality of processing units by one processor, first, as represented by a computer of a client, a server, and the like, there is an aspect in which one processor is configured by a combination of one or more CPUs and software and this processor functions as a plurality of processing units. Second, as represented by a system on chip (SoC) or the like, there is an aspect of using a processor that realizes the function of the entire system including the plurality of processing units by one integrated circuit (IC) chip. As described above, as the hardware structures, the various processing units are configured by using one or more of the various processors described above.

Further, as the hardware structures of these various processors, more specifically, it is possible to use an electrical circuit (circuitry) in which circuit elements, such as semiconductor elements, are combined.

The supplementary notes of the present disclosure will be described below.

### Supplementary Note 1

An information processing apparatus comprising at least one processor,
in which the processor is configured to:
   specify a first section and a second section in which a detection target of movement of a subject is different, based on a measurement value of the movement of the subject measured by a device that is mountable on the subject;
   acquire at least one feature amount representing a feature of a disease related to at least one of cognition or motion based on each of a measurement value of the first section and a measurement value of the second section; and
   acquire a determination result of the disease based on the feature amount and a predetermined determination criterion.

### Supplementary Note 2

The information processing apparatus according to supplementary note 1,
in which the first section is a section in which movement of an entire body of the subject is a detection target, and the second section is a section in which movement of a head of the subject is a detection target.

### Supplementary Note 3

The information processing apparatus according to supplementary note 2,
in which the measurement value includes a change in an acceleration of the subject in a vertical downward direction, and
the processor is configured to derive landing information indicating that the subject has landed during movement based on the change in the acceleration, and specify a walking section of the subject as the first section and specify a non-walking section of the subject as the second section based on continuity of the landing information.

### Supplementary Note 4

The information processing apparatus according to any one of supplementary notes 1 to 3,
in which the measurement value includes an acceleration of a head in a front-rear direction in the second section, and
the processor is configured to acquire the feature amount representing the feature of the disease related to the cognition based on the acceleration of the head in the front-rear direction in the second section.

### Supplementary Note 5

The information processing apparatus according to any one of supplementary notes 1 to 4,
in which the measurement value includes a rotation speed of a head in the second section, and
the processor is configured to acquire the feature amount representing the feature of the disease related to the motion based on the rotation speed of the head in the second section.

### Supplementary Note 6

The information processing apparatus according to any one of supplementary notes 1 to 5,
in which the measurement value includes movement in a left-right direction of the subject in the first section, and
the processor is configured to acquire the feature amount representing the feature of the disease related to the cognition based on the movement in the left-right direction of the subject in the first section.

### Supplementary Note 7

The information processing apparatus according to any one of supplementary notes 1 to 6,
in which the measurement value includes movement of the subject in a front-rear direction in the first section, and
the processor is configured to acquire the feature amount representing the feature of the disease related to the motion based on the movement of the subject in the front-rear direction in the first section.

### Supplementary Note 8

The information processing apparatus according to any one of supplementary notes 1 to 7,
in which the processor is configured to derive the determination criterion.

### Supplementary Note 9

The information processing apparatus according to any one of supplementary notes 1 to 8,
in which the determination criterion is a reference value for distinguishing between the disease and a non-disease.

### Supplementary Note 10

The information processing apparatus according to any one of supplementary notes 1 to 8,
in which the determination criterion is a feature amount distribution estimated based on a plurality of the feature amounts representing the feature of the disease.

### Supplementary Note 11

The information processing apparatus according to any one of supplementary notes 1 to 8,
in which the determination criterion is an identification model that has been trained to output a score representing a possibility of the disease in response to an input of the feature amount.

### Supplementary Note 12

The information processing apparatus according to any one of supplementary notes 1 to 11,
in which the disease is at least one of mild cognitive impairment, dementia, or Parkinson's disease.

### Supplementary Note 13

The information processing apparatus according to any one of supplementary notes 1 to 12,
in which the device includes an acceleration sensor and an angular velocity sensor.

### Supplementary Note 14

The information processing apparatus according to any one of supplementary notes 1 to 13,
in which the device includes an electrooculography sensor.

### Supplementary Note 15

An information processing method comprising, via a computer:
specifying a first section and a second section in which a detection target of movement of a subject is different, based on a measurement value of the movement of the subject measured by a device that is mountable on the subject;
acquiring at least one feature amount representing a feature of a disease related to at least one of cognition or motion based on each of a measurement value of the first section and a measurement value of the second section; and
acquiring a determination result of the disease based on the feature amount and a predetermined determination criterion.

### Supplementary Note 16

An information processing program causing a computer to execute:
a procedure of specifying a first section and a second section in which a detection target of movement of a subject is different, based on a measurement value of the movement of the subject measured by a device that is mountable on the subject;
a procedure of acquiring at least one feature amount representing a feature of a disease related to at least one of cognition or motion based on each of a measurement value of the first section and a measurement value of the second section; and
a procedure of acquiring a determination result of the disease based on the feature amount and a predetermined determination criterion.

What is claimed is:

1. An information processing apparatus comprising at least one processor,
wherein the processor is configured to:
specify a first section and a second section in which a detection target of movement of a subject is different, based on a measurement value of the movement of the subject measured by a device that is mountable on the subject;
acquire at least one feature amount representing a feature of a disease related to at least one of cognition or motion based on each of a measurement value of the first section and a measurement value of the second section; and
acquire a determination result of the disease based on the feature amount and a predetermined determination criterion.

2. The information processing apparatus according to claim 1,
wherein the first section is a section in which movement of an entire body of the subject is a detection target, and the second section is a section in which movement of a head of the subject is a detection target.

3. The information processing apparatus according to claim 2,
wherein the measurement value includes a change in an acceleration of the subject in a vertical downward direction, and
the processor is configured to derive landing information indicating that the subject has landed during movement based on the change in the acceleration, and specify a walking section of the subject as the first section and specify a non-walking section of the subject as the second section based on continuity of the landing information.

4. The information processing apparatus according to claim 1,
wherein the measurement value includes an acceleration in a front-rear direction of a head in the second section, and
the processor is configured to acquire the feature amount representing the feature of the disease related to the cognition based on the acceleration in the front-rear direction of the head in the second section.

5. The information processing apparatus according to claim 1,
wherein the measurement value includes a rotation speed of a head in the second section, and
the processor is configured to acquire the feature amount representing the feature of the disease related to the motion based on the rotation speed of the head in the second section.

6. The information processing apparatus according to claim 1,
wherein the measurement value includes movement in a left-right direction of the subject in the first section, and
the processor is configured to acquire the feature amount representing the feature of the disease related to the cognition based on the movement in the left-right direction of the subject in the first section.

7. The information processing apparatus according to claim 1,
wherein the measurement value includes movement of the subject in a front-rear direction in the first section, and
the processor is configured to acquire the feature amount representing the feature of the disease related to the motion based on the movement of the subject in the front-rear direction in the first section.

8. The information processing apparatus according to claim 1,
wherein the processor is configured to derive the determination criterion.

9. The information processing apparatus according to claim 1,
wherein the determination criterion is a reference value for distinguishing between the disease and a non-disease.

10. The information processing apparatus according to claim 1,
wherein the determination criterion is a feature amount distribution estimated based on a plurality of the feature amounts representing the feature of the disease.

11. The information processing apparatus according to claim 1,
wherein the determination criterion is an identification model that has been trained to output a score representing a possibility of the disease in response to an input of the feature amount.

12. The information processing apparatus according to claim 1,
wherein the disease is at least one of mild cognitive impairment, dementia, or Parkinson's disease.

13. The information processing apparatus according to claim 1,
wherein the device includes an acceleration sensor and an angular velocity sensor.

14. The information processing apparatus according to claim 1,
wherein the device includes an electrooculography sensor.

15. An information processing method comprising, via a computer:
specifying a first section and a second section in which a detection target of movement of a subject is different, based on a measurement value of the movement of the subject measured by a device that is mountable on the subject;
acquiring at least one feature amount representing a feature of a disease related to at least one of cognition or motion based on each of a measurement value of the first section and a measurement value of the second section; and
acquiring a determination result of the disease based on the feature amount and a predetermined determination criterion.

16. A non-transitory computer-readable storage medium that stores an information processing program causing a computer to execute:
a procedure of specifying a first section and a second section in which a detection target of movement of a subject is different, based on a measurement value of the movement of the subject measured by a device that is mountable on the subject;
a procedure of acquiring at least one feature amount representing a feature of a disease related to at least one of cognition or motion based on each of a measurement value of the first section and a measurement value of the second section; and
a procedure of acquiring a determination result of the disease based on the feature amount and a predetermined determination criterion.
